(19) United States
(12) Patent Application Publication
(10) Pub. No.: US 2025/0288669 A1
(43) Pub. Date: Sep. 18, 2025

(54) HUMAN T-CELL ACUTE LYMPHOBLASTIC LEUKEMIA CELL LINE & APPLICATIONS FOR TREATING CANCER

(71) Applicant: INmune Bio Inc., Boca Raton, FL (US)

(72) Inventors: Mark Lowdell, London (GB); Raymond J. Tesi, Delray Beach, FL (US); Joshua S. Schoonover, Boca Raton, FL (US)

(73) Assignee: INmune Bio Inc., Boca Raton, FL (US)

(21) Appl. No.: 18/998,789

(22) PCT Filed: Aug. 3, 2023
(86) PCT No.: PCT/US2023/029426

§ 371 Date: Jan. 27, 2025

Related U.S. Application Data
(60) Provisional application No. 63/394,862, filed on Aug. 3, 2022.
(60) Provisional application No. 63/417,514, filed on Oct. 19, 2022.

(51) Int. Cl. A61K 40/11; A61K 35/17; A61P 35/02; C12N 5/0783; C12N 5/09; C12N 13/00

(52) U.S. Cl. CPC A61K 40/11; A61K 35/17; A61P 35/02; C12N 5/0636; C12N 5/0693; C12N 13/00; A61K 2239/38; A61K 2239/48

(57) ABSTRACT

A novel human T-cell acute lymphoblastic leukemia (T-ALL) cell line called INB16 (ATCC Deposit no. PTA-125809) induces memory like function on natural killer cells upon contact therewith, which memory like natural killer cells have demonstrated ability to identify and kill cancer cells, including hematologic and solid tumor cells. Useful applications of the INB16 cell line include research, a cancer therapeutic agent comprising replication incompetent INB16 cells and/or membrane portions thereof for in vivo administration and restoring function of a patient's own NK cells, and related methods of treating cancer.

### CROSS-REFERENCE TO RELATED APPLICATIONS

This application claims benefit of priority with U.S. provisional application Ser. No. 63/394,862, filed 3 Aug. 2022, and Ser. No. 63/417,514, filed 19 Oct. 2022; the entire contents of each of which is hereby incorporated by reference for all purposes.

### TECHNICAL FIELD

The present invention relates to the field of immortal human T-cell acute lymphoblastic leukemia cells, and related compositions and methods for treating cancer.

### BACKGROUND ART

CTV-1 (Product No. ACC-40, DSMZ), hereinafter “CTV-1”, is a human monocytoid leukemic cell line established from peripheral blood of a 40-year-old female patient with relapsed acute monoblastic leukemia circa 1982. Peripheral blood (10 mL) was obtained from this patient by venipuncture. At this time, 15×10⁴/mm² and more than 90% of these cells were blast. The leukemic cells were first separated by Ficoll-Hypaque gradient centrifugation and then washed three times with RPMI-1640 medium. They were then suspended in a flask in the same medium supplemented with 20% heat-inactivated fetal bovine serum. The suspended cells were incubated at 37° in a humidified atmosphere with 5% CO₂ and fed twice per week. From the beginning, the cells showed slow but definite multiplication. After 4 weeks, the cells grew vigorously in suspension and were serially transferred every 3-4 days. These cells grew best at a concentration of 2×10⁵ cells/mL, and the doubling time was about 36 hours. See Chen. 1984.

Other human leukemia cell lines include K562, HL-60, KG-1, U-937, PL-21, KCL-22, BV173, THP-1, RC-2A, and P31/Fujioka.

T-cell acute lymphoblastic leukemia is an aggressive type of leukemia in which too many T-cell lymphoblasts are found in the bone marrow and blood. T-cell acute lymphoblastic leukemia is also called precursor T-lymphoblastic leukemia and T-cell acute lymphocytic leukemia.

### SUMMARY OF INVENTION

### Technical Problem

Human leukemic cells are generally very difficult to proliferate in vitro. Leukemic colonies usually undergo terminal differentiation, and subculturing is usually not successful for more than two or three passages.

While there are a number of established leukemic cell lines available for research, there is a need for additional leukemic cell lines to enhance understanding of the various leukemias and for developing therapeutics and therapeutic strategies for treatment of disease.

Moreover, novel biopharmaceutical compositions that are effective in treating cancer are in continued need and high demand.

Still further, methods which incorporate or make use of such novel biopharmaceutical compositions are also in continued need and high demand.

### Solution to Problem

A novel human T-acute lymphoblastic leukemia cell referred to herein as “INB16” (ATCC Deposit no. PTA-125809) was isolated and immortalized in a cell line useful for cancer research and development of biopharmaceutical anti-cancer therapeutics.

Replication incompetent INB16 cells, membrane portions thereof, and combinations of replication incompetent INB16 cells and membrane portions, were prepared to form NK cell priming agents useful for treating cancer.

In various embodiments, the invention incorporates or makes use of cells of a novel INB16 cell line for purposes of research and development of therapeutics and therapeutic strategies for treatment of cancer.

### Advantageous Effects of Invention

When made replication incompetent, cells from an INB16 cell line, and/or membrane portions of such cells, can be used as an agent for priming natural killer cells of a subject in vivo, such that the primed NK cells may become useful to surveil, identify and kill cancer cells. In this regard, the INB16 cells have a unique biological signature that achieves in vivo NK cell priming for promoting NK cell function in a patient suffering from cancer.

Surprisingly, NK cells primed by replication incompetent INB16 cells demonstrate a memory-like phenotype, meaning that the NK cells remain primed and therapeutically active for a prolonged duration.

NK cell priming by replication incompetent INB16 cells can be achieved without the use of cytokines, thereby mitigating downsides of potential cytokine storm.

The effects of a single course of therapy can last for at least four months.

NK cell priming by replication incompetent INB16 cells works in hostile tumor microenvironment (TME) of solid tumors. The TME is hypoxic (low oxygen level). Immune cells generally do not function in the hypoxic TME. NK cells primed by a cell preparation including replication incompetent INB16 cells function in a hypoxic TME.

Avidity of NK cells binding to tumor cells is increased in such NK cells primed by replication incompetent INB16 cells. In other words, a cell preparation including replication in competent INB16 cells was shown to enhance avidity of NK cells for binding tumor cells.

Persistence is increased in such NK cells primed by replication incompetent INB16 cells.

A cell preparation comprising replication incompetent INB16 cells was shown to activate critical genes and pathways for enhanced NK cell function and survival. INB16 upregulates 522 genes distinct from IL2, including CD70, CXCL10, and Stat5. Additionally, INB16 upregulates 1461 proteins distinct from IL15, including TIMM29, and USP20.

### BRIEF DESCRIPTION OF DRAWINGS

FIG. 1 shows a plot of % activated NKG2D NK cells and % blast cells in a patient treated with a cell preparation comprising replication incompetent INB16 cells.

FIG. 2 shows in vitro killing of NK cells obtained from a patient treated with a cell preparation comprising replication incompetent INB16 cells.

FIG. 3 shows a table indicating the effect of healthy donor NK cells on various tumor cell lines, both with and without INB16 treatment.

FIG. 4 shows a table reflecting the effect of NK cells obtained from cancer patients treated with riINB16 cells on various cancer cell lines in an in vitro assay.

FIG. 5 shows a plot illustrating target cell lysis-associated proteins that are changed in response NK cells treated with IL-15, INB16, or no treatment.

FIG. 6 shows a plot illustrating mitochondrial survival-associated proteins that are changed in response NK cells treated with IL-15, INB16, or no treatment.

FIG. 7 shows a plot illustrating avidity against SKOV3 for each of rNK, NK-IL-2, NK-IL-15, and riINB16-primed NK cells.

FIG. 8 shows a plot representing percent cytolysis of rNK and TP-NK cells in a hypoxic environment simulating the TME.

FIG. 9 shows a plot indicating riINB16-primed NK cells from a MDS patient demonstrate restored ability to kill MDS cells in vitro.

### DESCRIPTION OF EMBODIMENTS

The following description, for purposes of explanation and not limitation, includes certain details and descriptions of preferred embodiments of the invention as set forth in the claims. One with skill in the art will recognize that these details and descriptions are not exhaustive, and taken together with the ordinary level of skill in the art, as of the date of this submission, aid the reviewer in understanding how to make and use the inventive concepts herein disclosed and claimed. Nothing in these details and descriptions shall be construed of limiting the spirit and scope of the invention.

Prior to the observations and discoveries presented herein, it was established that human patients suffering from various forms of cancer, e.g. leukemia, usually have natural killer (NK) cells that are less functional than those of healthy individuals, which led the scientific community to assume an underlying issue in progression of cancer was one of immune cell dysfunction, and more particularly, defective NK cells. For example, it was observed that NK cells in relapsed patients did not have the ability to kill residual cancer cells.

More recently, in the course of research aimed at understanding the role of NK cells in the innate immune response to cancer, a number of observations and discoveries were made that suggest the problem is not in the NK cell, but in the environment from within which the NK cell exists.

For example, it was observed that although patients with acute myeloid leukemia (AML) had NK cells that were less active than those of healthy individuals, as evidenced by in vitro cell killing assays, the NK cells from AML patients after chemotherapy were able to kill AML cancer cells. Further investigation identified that as-between surviving AML patients (those surviving two or more years) versus those AML patients that relapsed after chemotherapy treatment, no differences in NK function were observed, meaning the NK cells seemed to be equally functional as-between surviving and relapsing post-chemotherapy AML patients. Subsequent to these observations, further analysis was done on the AML tumor cells of various patients and their ability to induce NK cell killing of NK-resistant cells from the RAJI cell line, which revealed that the NK cell, or function/dysfunction thereof, wasn't the difference in survivors, but the patient's own leukemia cell variant was directly involved in whether resulting NK cells could kill RAJI cells or not in vitro, or whether the cancer cells of the patient could induce the patient's own NK cells to kill residual cancer in the patient post-chemotherapy treatments. It was therefore discovered that some cancer cells (derived from the survivors) appear to have a biological effect on a patient's NK cells, enabling the NK cells to surveil, recognize and kill cancer cells: whereas other cancer cells, incubated with the same NK cells, do not result in cancer cell killing.

This discovery, that certain cancer cells can impact the function of a patient's NK cells, either inducing an effector state or not, led to a series of experiments testing whether different cancer cells have ability to change NK cells from a resting (non-effector) state to an active (effector) state.

It was further discovered that CTV-1 cells are surprisingly capable of converting resting NK cells to activated NK cells in response to the NK-resistant RAJI cell line in vitro. This work led to a method of treating cancer that includes in vitro activation of NK cells by CTV-1 co-culture and subsequent infusion into a patient in need thereof. See, for example, U.S. Pat. No. 8,257,970.

While CTV-1 was shown to be successful for cancer killing in vitro, and there was some success in human patients, there are draw backs such as high costs of in vitro culture, manufacturing, and subsequent transfusion as a therapeutic strategy for treating cancer.

Based on these observations, a method of in vivo priming of NK cells was conceived, including the steps of inactivating CTV-1 derived leukemia cells to make them replication incompetent and administering the replication incompetent CTV-1 cells directly to the patient in vivo by infusion or other acceptable route of administration. See, for example, U.S. Pat. No. 10,758,567.

While in vivo priming of NK cells using cells from a CTV-1 cell line appeared to be efficacious for treating residual cancer, it was not clear whether other leukemia cells could be used in a similar manner, that is, made replication incompetent and administered to a patient for in vivo priming of the patient's own NK cells thereby enabling the NK cells to kill residual cancer. Another leukemia cell line was desirable to test the open hypothesis.

### Preparation of Cell Lines

Cell lines may be produced according to established methodologies known to those skilled in the art. In general, cell lines are produced by culturing primary cells derived from a patient until immortalized cells are spontaneously generated in culture.

These cells are then isolated and further cultured, to produce clonal cell populations or cells exhibiting resistance to apoptosis.

For example, T-ALL cells may be isolated from peripheral blood drawn from a patient suffering from T-ALL. The cells may be washed, and optionally immunotyped in order to determine the type(s) of cells present. Subsequently, the cells may be cultured in a medium, such as a medium containing IL-4 or other replication stimulating factor. Advantageously, all or part of the medium is replaced one or more times during the culture process. Cell lines may be isolated thereby and will be identified by increased growth in culture.

In accordance with the foregoing, herein described are cells from the T-cell acute lymphoblastic leukemia cell line INB16 (ATCC Deposit no. PTA-125809).

In one useful application of the INB16 cell line, a biopharmaceutical composition comprising cells and/or membrane portions of cells from an INB16 cell line (ATCC Deposit no. PTA-125809) is disclosed, wherein the cells and/or membrane portions of cells are rendered replication incompetent.

The cells from said INB16 cell line can be made or rendered replication incompetent by contacting the cells with mitomycin C.

Alternatively, the cells from the INB16 cell line can be rendered replication incompetent by first immobilizing said cells in a dry carbohydrate matrix and second dosing said cells while immobilized with a permeating ionizing radiation. Cells can be immobilized in a dry carbohydrate matrix by preparing a solution comprising a non-reducing sugar, such as but not limited to sucralose, and suspending the cells in the solution to form a suspension, wherein after suspending the suspension is dried according to a primary drying protocol incorporating temperature and pressure modulation in a lyophilizer to achieve gradual and simultaneous evaporation, sublimation and boiling of the suspension to remove water until there is less than 5% residual water content. An example of this preservation technique is disclosed in U.S. Pat. No. 9,469,835, issued Oct. 18, 2016. In some examples, the dose may comprise a permeated ionizing radiation dose and may be delivered by electron beam irradiation, gamma irradiation, or X-ray irradiation. The dose may comprise at least 2 Gy and up to 20 kGy or more.

In another aspect, a method for treating cancer is disclosed, the method comprising: administering to a patient having said cancer a therapeutically effective amount of a priming tumor cell preparation for priming natural killer cells of the patient in vivo, the priming tumor cell preparation including: cells and/or membrane portions thereof derived from an INB16 cell line (ATCC Deposit no. PTA-125809), wherein the cells and/or membrane portions of the priming tumor cell preparation are rendered replication incompetent to prevent proliferation in the patient: whereby said patient is treated. The cancer may comprises a solid tumor or a hematologic tumor. In an embodiment, the cancer may comprise acute myeloid leukemia (AML). In another embodiment, the cancer may comprise myelodysplastic syndrome. In preferred embodiments, the therapeutically effective amount of cells in a dose of the priming tumor cell preparation comprises one, two or three weekly administrations of 1×10⁸ cells, the cells being derived from an INB16 cell line and rendered replication incompetent.

### Example 1—STR Analysis of CTV-1 Cells

CTV-1 cells were obtained, short tandem repeat (STR) analysis was performed, and results of STR analysis were recorded.

In the following Table 1, STR analysis was performed against the CTV-1 cells using AmpFISTR Identifier Plus and AmpFISTR NGM kits (Applied Biosystems), which analyze 21 loci consistent with all major worldwide standards (including D21S11, CSF1PO, vWA, D8S1179, TH01, D18S51, D5S818, D16S539, D3S1358, D2S1338, TPOX, FGA, D7S820, D13S317, Amelogenin, D19S433, D10S1248, D22S1045, D2S441, DIS1656, and D12S391). Allele detection has been conducted by PCR amplification and subsequent capillary sequencing using an ABI PRISM 3100-Avant Genetic Analyzer (Applied Biosystems). Sequencing results were analyzed using Genemapper Software (Applied Biosystems).

TABLE 1
Results of STR on CTV-1

| Loci | Results | Loci | Results |
|---|---|---|---|
| D8S1179 | 12/13 | TPOX | 8/11 |
| D21S11 | 27/29 | D18S51 | 13/14 |
| D7S820 | 9/11 | AMEL | X/X |
| CSF1PO | 12/12 | D5S818 | 12/12 |
| D3S1358 | 16/16 | FGA | 22/25 |
| TH01 | 6/9.3 | D10S1248 | 14/14 |
| D13S317 | 12/12 | D22S1045 | 16/17 |
| D16S539 | 12/12 | D2S441 | 10/10 |
| D2S1338 | 17/20 | D1S1656 | 17.3/17.3 |
| D19S433 | 14/16 | D12S391 | 17/18 |
| vWA | 14/15 | | |

### Example 2—A Novel INB16 Cell Line

INB16 is a human T-cell acute lymphoblastic leukemia (T-ALL) cell line established from peripheral blood of a male patient with T-cell acute lymphoblastic leukemia (T-ALL).

INB16 cells were isolated in accordance with conventional techniques and suspended in RMPI-1640 medium, with L-Glutamine, and with 10% fetal bovine serum. Cells are seeded at 0.5×10⁶ cells/mL in static culture and harvested at (1.0-2.0)×10⁶ cells/mL. It is desirable to store cells at vapor phase liquid nitrogen.

INB16 cells were obtained, short tandem repeat (STR) analysis was performed, and results of STR analysis were recorded.

In the following Table 2, STR analysis was performed against the INB16 cells using AmpFISTR Identifier Plus and AmpFISTR NGM kits (Applied Biosystems), which analyze 21 loci consistent with all major worldwide standards (including D21S11, CSF1PO, vWA, D8S1179, TH01, D18S51, D5S818, D16S539, D3S1358, D2S1338, TPOX, FGA, D7S820, D13S317, Amelogenin, D19S433, D10S1248, D22S1045, D2S441, DIS1656, and D12S391). Allele detection has been conducted by PCR amplification and subsequent capillary sequencing using an ABI PRISM 3100-Avant Genetic Analyzer (Applied Biosystems). Sequencing results were analyzed using Genemapper Software (Applied Biosystems).

TABLE 2
Results of STR on INB-16

| Loci | Results | Loci | Results |
|---|---|---|---|
| D8S1179 | 9/15 | TPOX | 8/8 |
| D21S11 | 27/28 | D18S51 | 8/14 |
| D7S820 | 11/12/13 | AMEL | X/Y |
| CSF1PO | 9/10/12 | D5S818 | 12/12 |
| D3S1358 | 15/17 | FGA | 23/24/25 |
| TH01 | 9/9 | D10S1248 | 13/17/18 |
| D13S317 | 11/12 | D22S1045 | 16/17/18/19 |
| D16S539 | 9/14/15 | D2S441 | 10/11/14 |
| D2S1338 | 17/18 | D1S1656 | 14/15/16/17 |
| D19S433 | 13/14/16 | D12S391 | 19/20/21 |

The INB16 cell line is deposited with ATCC Patent Depository, 10801 University Boulevard, Manassas, Virginia 20110 USA in accordance with the Budapest Treaty as ATCC Deposit no. PTA-125809.

Key differentiating loci as-between CTV-1 and INB16 are noted at least in loci AMEL, D10S1248, D16S539, and D12S391. While both are leukemia cell lines, given the genetic distinctions between CTV-1 and INB16 cell lines as evidenced by the enclosed STR results, it was unknown whether INB16 cells will have the biological effects demonstrated with CTV-1, and thus additional experiments were necessitated.

### Example 3—In Vitro Tumor Killing by NK Cells Primed with Ri-INB16

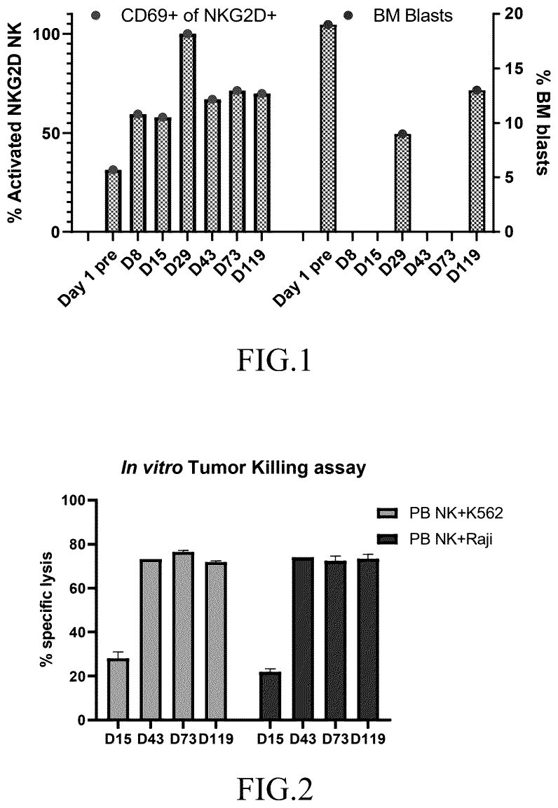

A 78-year-old male human subject with MDS was given a treatment comprising three administrations of replication incompetent INB16 (ri-INB16) cells, the first 3×10⁸ cell, and the second and third administrations each comprising 1×10⁸ cells. On days-1 (pre-treatment), +8, +15, +29, +43, +73, and +119 relative to the first treatment administration, blood from the patient was obtained and NK cells were isolated therefrom. The patient's percent of activated NK cells were identified as those with biomarkers CD69+ and NKG2D+. FIG. 1 shows results, where the patient presented about 30% activated NK cells pre-treatment, and at least 60% activated NK cells post treatment up to at least day +119. Thus, the data indicates that a treatment regime comprising administration by infusion of replication incompetent INB16 cells provided an increase in activated NK cells in the patient's blood beginning immediately post-treatment and continuing at least +119 days. In addition, also reflected in FIG. 1, the patient showed a sustained reduction in blast cells from day −1 (pre-treatment, 100%) to day +29 (45%) and day +119 (65%).

Next, the NK cells isolated from the patient were cultured with NK-resistant tumor cell lines K562 and RAJI to assess the effects of ri-INB16 primed NK cells for killing cancer. In vitro cancer cell % specific lysis was measured at days +15, +43, +73, and +119. FIG. 2 shows immediate cancer killing at day +15, which appears to peak by day +43, and is consistent through at least day +119. These results indicate that NK cells primed with ri-INB16 cells in vivo are capable of killing NK-resistant cancer cells (K562 and RAJI) in vitro. But for the ri-INB16 priming, these NK cells would be incapable of killing the NK-resistant cancers cells.

### Example 4—Solid Tumor Killing of INB16-timlNK Cells Versus Resting NK Cells

It was surprisingly discovered that INB16 tumor induced memory-like NK (INB16-timlNK) cells significantly improved tumor cell killing in in vitro assays. Experimental data shows INB16-timlNK cells are efficacious against NK-resistant cell lines, including DU145, 786O, ACHN, SKOV3, H3, and C17. From this data a method of treating a solid tumor in a subject is proposed, the method comprising administering to the subject a therapeutically effective amount of an NK-cell priming agent, the NK-cell priming agent comprising replication-incompetent cells and/or membrane portions thereof, the cells and/or membrane portions being derived from an INB16 cell line.

Tumor induced memory-like NK (timlNK) cells are a phenotype of NK cells that demonstrate ability to lyse NK-resistant tumor cells and demonstrate memory-like persistence. One with skill in the art will appreciate that timlNK cells are distinct from cytokine induced memory-like NK (cimlNK) cells, which demonstrate ability to lyse certain NK-resistant tumor cells, and demonstrate memory-like persistence, though less persistence than INB16-timlNK cells. Additionally, INB16-timlNK cells present a distinct proteomics signature compared to cimlNK cells, including upregulation of at least one hundred forty-one expressed proteins. For clarity, cimlNK cells are generally made by culturing resting NK cells with cytokines, especially IL-2, IL-15 or IL-12/15/18.

The INB16-timlNK cells can be produced in vitro by exposing resting NK cells to INB16, or they may be in vivo from in vivo exposure of resting natural killer (rNK) cells to INB16 (replication incompetent INB16) or a preparation comprising membrane fragments of INB16 cells; either way by contacting the rNK cells with replication incompetent INB16 cells or membrane portions thereof. The resulting INB16-timlNK cells are potent for killing cancer.

In this example, INB16-timlNK cells were created in vitro by culturing human resting NK cells obtained from healthy donors with replication incompetent cells of an INB16 cell line that were inactivated by mitomycin C. The resulting NK cells are referred to here as INB16-timlNK cells. Another option for creating replication incompetent NK cells is radiation; however, care should be taken to ensure NK cell priming ligands are not destroyed by the dose of radiation. In order to achieve proper replication incompetency using radiation, one must not over-energize the cells, as this could destroy epitopes useful for NK cell priming. For these reasons, mitomycin C is a preferred means for rendering tumor cells replication incompetent for use in the embodiments as described herein.

In another example, not illustrated here, replication incompetent INB16 cells can be administered directly to a patient via intravenous infusion, with a dose between 1.0×10⁶ and 1.0×10⁹ cells per treatment, and one treatment each week for three consecutive weeks. A preferred dose of replication incompetent INB16 cells is 1.0×10⁸ for human subjects.

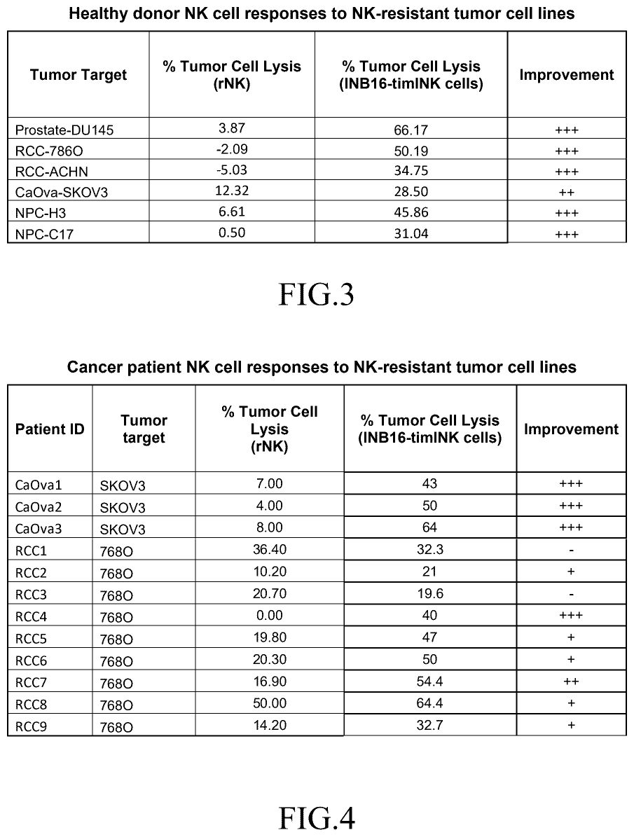

With this background, we turn to the instant example associated with FIG. 3, wherein rNK cells were obtained from healthy donors and divided into two allocations, one remaining as rNK (not exposed to INB16 cells), and the other cultured with replication incompetent cells of an INB16 cell line (ri-INB16) to generate INB16 tumor induced memory like NK (INB16-timlNK) cells. The rNK cells or INB16-timlNK cells were introduced into solid tumor cell lines in vitro and percentage NK lysis was determined in accordance with conventional techniques. The solid tumor cell lines include DU145 (prostate cancer), 7860 (renal cell carcinoma), ACHN (renal cell carcinoma), SKOV3 (ovarian cancer), H3 (nasopharyngeal cancer), and C17 (nasopharyngeal cancer). As seen in FIG. 3, INB16-timlNK cells outperformed rNK in each of the tumor cell models. Improvement of 0-200% (+), 201%-500% (++), and greater than 501% (+++) are denoted in the column labeled “Improvement”. The INB16-timlNK cells demonstrated enhanced ability to kill these tumor cell lines.

Thus, a method for treating cancer may comprise administering to a subject in need thereof a therapeutically effective amount of a NK cell priming agent comprising replication incompetent INB16 cells, wherein the subject is treated, and wherein the cancer comprises a solid tumor selected from prostate cancer, renal cell carcinoma, ovarian cancer, or nasopharyngeal cancer.

FIG. 4 shows performance of NK cells obtained from human cancer patients, incubated with replication incompetent INB16 cells, and introduced into SKOV3 (ovarian cancer patients) or 768O (renal cell carcinoma patients) cell lines. As illustrated, timlNK cells significantly outperformed the patient's rNK cells in killing tumor cells in vitro. The data is represented in percent tumor cell lysis for each of the rNK and timlNK cells, and Improvement is denoted in FIG. 2 as −200%-0 (−), 0-200% (+). 201%-500% (++), and greater than 501% (+++).

### Example 5—INB16-timlNK Cells are Distinguished from cimlNK Cells

More than 1,500 proteins were identified that are upregulated in INB16-timlNK cells, and subsequent analysis compared them to NK cells primed with a cytokine cocktail of IL-12, IL-15 and IL-18 (to generate cimlNK cells). Of the 250 most upregulated proteins, 141 are completely unique to INB16-timlNK cells and are not upregulated by the cytokines IL-12, IL-15 and IL-18. Many of these unique proteins are involved in cell survival and the enhanced metabolism likely to protect INB16-timlNK cells in the TME.

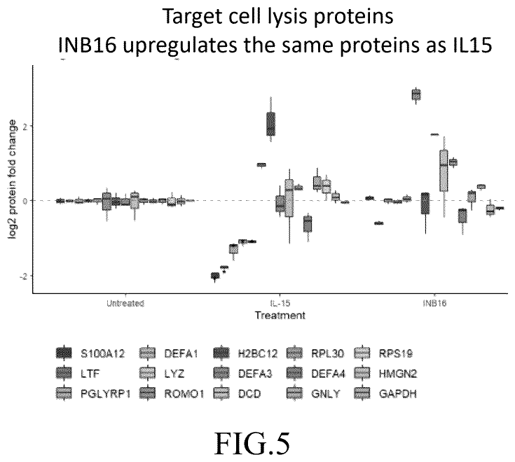

FIG. 5 shows a plot of target cell lysis associated proteins as measured from untreated resting NK cells, IL-15 cytokine induced memory like NK (IL15-cimlNK) cells, and INB16-timlNK cells. Here, untreated rNK cells demonstrate negligible log 2 protein fold change, whereas both IL15-cimlNK and INB16-timlNK cells demonstrate protein fold changes, specifically: S100A12, LTF, PGLYRP1, DEFA1, LYZ, ROMO1, H2BC12, DEFA3, DCD, RPL30, DEFA4, GNLY, RPS19, HMGN2, and GAPDH. These results indicate that INB16-timlNK cells are similar to IL15-cimlNK in terms of expression of target cell lysis-associated proteins.

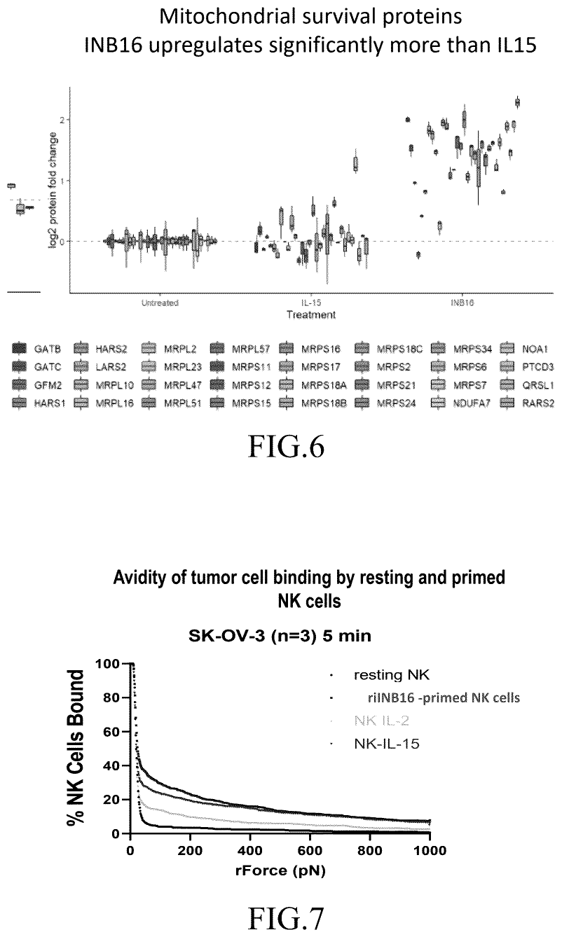

FIG. 6 shows a plot of mitochondrial survival associated proteins as measured from untreated resting NK cells, IL-15 cytokine induced memory like NK (IL15-cimlNK) cells, and INB16-timlNK cells. Here, untreated rNK cells and IL15-cimlNK demonstrate negligible log 2 protein fold change, whereas INB16-timlNK cells demonstrate meaningful protein fold changes, specifically: GATB, GATC, GFM2, HARS1, HARS2, LARS2, MRPL10, MRPL16, MRPL2, MRPL23, MRPL47, MRPL51, MRPL57, MRPS11, MRPS12, MRPS15, MRPS16, MRPS17, MRPS18A. MRPS18B, MRPS18C, MRPS2, MRPS21, MRPS24, MRPS34, MRPS6, MRPS7, NDUFA7, NOA1, PTCD3, QRSL1, and RARS2. These results indicate that INB16-timlNK cells are superior to IL15-cimlNK and rNK cells in terms of expression of mitochondrial survival-associated proteins. These results are believed to be related to the observed improvement of NK cell persistence observed in INB16-timlNK cells compared to rNK, and IL15-cimlNK.

For purposes herein, the terms “riINB16-primed NK cells” and “INB16-timlNK cells” are interchangeable. The former is used to indicate replication incompetent INB16 cells are used to prim the resulting NK cells, whereas the latter is used to indicate INB16 is used to induce a phenotype of tumor induced memory like NK cells (INB16 being the tumor cell that induces the NK phenotype).

### Example 6—Replication Incompetent INB16 Cell Preparation for Treating Cancer

NK cells can have potent anti-tumor responses and recently highlighted potential for memory-like functions, but the best approach to generate memory-like NK (mlNK) cells has so far remained unclear. Priming of NK cells with the pharmaceutical-grade, replication incompetent tumor cell product derived from INB16 cells generates tumor-induced mlNK (TIML-NK) cells with enhanced cytokine production and cytotoxicity against multiple NK-resistant tumor target cell lines in vitro, analogous to previously reported cytokine-primed memory-like NK cells. In addition, proteomic profiling of TIML-NK cells revealed differential abundance of proteins involved in promoting mitochondrial survival and function as well as critical nutrient receptors, which may provide a unique benefit of NK cell activation whilst sparing mitochondrial damage typically associated with cytokine-mediated activation. NK cell priming by a cell preparation comprising replication-incompetent INB16 cells increased NK glycolysis and oxidative phosphorylation whilst enhancing mitochondrial respiratory capacity and maintaining glycolytic reserve.

A cell preparation comprising INB16 cells was rendered replication incompetent by contacting the cells in vitro with mitomycin C.

Four human patients were each treated with a cycle of three, weekly infusions of 1×10⁸ replication incompetent INB16 cells. All four patients received each of the three doses without incident and without side effects.

Patient 1 was a 78-year-old man with a three year history of refractory MLD MDS who was transfusion and platelet dependent and required G-CSF. Within seven days of first infusion, over 50% of his peripheral blood NK cells activated and this increased with each dose. By day +29, 72% of his NK cells were activated and this remained above 68% at day +119 when monitoring ended. His ECOG status fell from 2 pre-treatment to 0 at day +119 and his RAD-1 ctDNA from 45 to 38. Analysis of systemic cytokines showed increases in MIP-1a/b, TNF-α and sIL2R, which paralleled the changes in percentages of CD69+ NK cells. IL6 levels peaked after the second infusion, but levels remained low and there was no evidence of CRS. At one year follow-up he remained well, platelet and G-CSF independent, with reduced transfusion requirements and had resumed playing sport with friends.

Patients 2, 3, and 4 were all multiply relapsed and refractory AML patients: two of whom had relapsed after HLA-mismatched HSCT. All showed rapid activation of peripheral NK cells post infusion with sustained activity throughout follow-up. In all cases these activated NK cells killed NK-resistant tumor cells in vitro without additional activation.

Patient 2 was a neutropenic 21-year-old female with 70% a mixed chimera and refractory AML (M2) post VUD HSCT at time of treatment. Within 1 month of initiation of INKmune treatment she was discharged with PMN>500 and her mixed chimera resolved to full donor chimera. At day +140 post treatment with a cell preparation comprising relication-incompetent INB16 cells her bone marrow NK cells remained highly activated (>60% CD69+. K562 and Raji lysis of >70%). Her AML recurred 4 months post infusion and she died from relapsed disease 8 months after treatment.

Patient 3 was a 21-year-old male with refractory AML (M6) after two failed HSCT (haplo m/m and VUD). He showed the same rapid generation of activated NK cells in vivo but to a lesser degree (maximum 35% CD69+, sustained increased lysis of K562 but no lysis of Raji cells) and no evidence of clinical improvement.

Patient 4, a multiply relapsed AML patient has just been treated and has shown peripheral NK proliferation and maturation at day +8. The subject experienced no adverse effects post 1st and 2nd doses.

Infusions of the cell preparation comprising replication incompetent INB16 cells proved safe and led to rapid and sustained activation of peripheral NK cells with concomitant increases in relevant systemic cytokines. The TIML-NK have a distinct phenotypic, proteomic and metabolomic signature which mirrors the activity in vitro and in vivo.

This is the first time memory-like NK cells have been generated in vivo without the need for cytokine induction or support and these findings inform the development of more optimal NK cell-based immunotherapeutic strategies for cancer.

### Example 7—riINB16-Primed NK Cells Demonstrate Increased Tumor Binding Vidity

Various NK cell preparations were obtained, including resting NK (rNK) cells, replication incompetent INB16 (riINB16)-primed NK cells, IL-2 primed NK (NK-IL-2) cells, and IL-15 primed NK (NK-IL-15) cells. Each of these preparations was investigated for % NK cells bound versus rForce (pN) using the LUMICKS Cell Avidity Analyzer, and results were observed and recorded. Specifically, ultrasound is used to exert forces completely contactless to NK cells in a microfluidic chip. The technology is based on ultrasound that is used to exert forces inside a microfluidic chip. Tumor cells are seeded in the chip and anchored to the bottom. NK cells are then introduced and allowed to interact with the tumor cell. By switching on the ultrasound, acoustic forces are applied without even touching the NK cells and those forces are directed upward. An NK cell displaying a strong tumor specificity will require a high force to be displaced. An NK cell which is not specific to any tumor antigen will instead require a weak force to be displaced, because no immune synapse is formed in this scenario.

FIG. 7 shows a plot of % NK Cells Bound versus rForce (pN) indicating the order of stronger avidity for tumor cell binding (SKOV3), whereas riINB16-primed NK cells have the strongest avidity, followed by NK-IL-15 cells, NK-IL-2 cells, and rNK cells, in order.

### Example 8—riINB16-Primed NK Cells Function in Hypoxic Environment

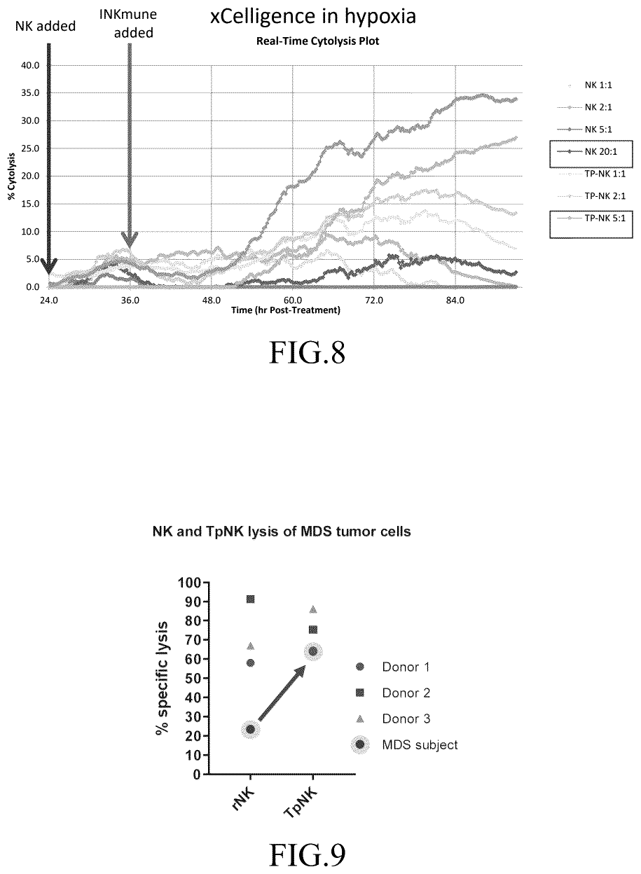

FIG. 8 shows % cytolysis versus time (hours post treatment) of NK cells before and after treatment with a cell preparation comprising riINB16 cells, which is done in a hypoxic environment. Here, untreated (non-primed) NK cells demonstrate markedly lower % cytolysis compared to tumor-primed NK (TP-NK) cells that were primed with riINB16. Moreover, as the ratio of riINB16 to NK cells was increased (1:1, 2:1, 5:1), % cytolysis also increased. This data indicates that riINB16-primed NK cells demonstrate increased cytolysis in a dose dependent manner. Because the tumor microenvironment (TME) is hypoxic, this data indicates that tumor primed NK cells resulting from contact with riINB16 are functional in a hypoxic environment, such as in the TME.

### Example 9—riINB16-Primed NK Cells Restore Tumor-Killing to Dysfunctional NK Cells

NK cells from three healthy human donors and from one human patient with MDS were obtained from peripheral blood. Tumor cell killing was investigated in vitro using an MDS tumor cell line and the corresponding NK cell ability to lyse the MDS cells. Resting NK (rNK) cells were evaluated against tumor primed NK (TPNK) derived from riINB16 cells. FIG. 9 shows rNK cells from healthy donors were able to lyse the MDS cells in vitro; however rNK cells from the MDS subject were unable to kill the MDS cells. In contrast, all NK cells were contacted with replication incompetent INB16 (riINB16) to form TPNK cells, and when presented to MDS cells in vitro, all NK cells were able to lyse the MDS cells, including those associated with the MDS subject. This data suggests priming a MDS subject's NK cells with a cell preparation comprising riINB16 cells results in primed NK cells capable of contacting and killing MDS cells.

### INDUSTRIAL APPLICABILITY

Cells from an INB16 cell line are useful for research and development of therapeutics and therapeutic strategies for treating cancer.

Compositions comprising replication incompetent cells and/or membrane portions from an INB16 cell line are useful as therapeutics and use in therapeutic strategies for treating cancer.

Methods of treating patients suffering from cancer that incorporate or make use of compositions comprising replication incompetent cells and/or membrane portions from an INB16 cell line are useful as part of therapeutic strategies for treating cancer.

These and other industrial applications will be apparent to one with skill in the art upon a review of the instant disclosure, including the claims and documents that are incorporated by reference.

### REFERENCE TO DEPOSITED BIOLOGICAL MATERIAL

The INB16 cell line is deposited with ATCC Patent Depository, 10801 University Boulevard, Manassas, Virginia 20110 USA in accordance with the Budapest Treaty as ATCC Deposit no. PTA-125809 first deposited 26 Jun. 2019 and confirmed viability on 5 Jul. 2019.

### CITATION LIST

The entire contents of each of the following references is hereby incorporated by reference for all purposes:
1. CHEN, P., CHIU, C., CHIOU, T., MAEDA, S., CHIANG, H., TZENG, C., SUGIYAMA, T., & CHIANG, B. N. (1984). ESTABLISHMENT AND CHARACTERIZATION OF A HUMAN MONOCYTOID LEUKEMIA CELL LINE, CTV-1. GANN Japanese Journal of Cancer Research, 75(8), 660-664. (Chen et. al., 1984).
2. Lowdell, Mark et al. (2022). In Vivo Generation of Memory-like NK Cells for the Treatment of AML and Myelodysplastic Syndrome; Early Clinical Applications of INKmune™. Blood. 140. 4590-4591. 10.1182/blood-2022-166175.
3. LOWDELL, MARK, U.S. Pat. No. 8,257,970, issued Sep. 4, 2012.
4. TESI, RAYMOND J. et al., U.S. Pat. No. 10,758,567, issued Sep. 1, 2020.
5. BRONSHTEIN, VICTOR, U.S. Pat. No. 9,469,835, issued Oct. 18, 2016.

What is claimed is:

1. Cells from the T-cell acute lymphoblastic leukemia cell line INB16 (ATCC Deposit no. PTA-125809).

2. A biopharmaceutical composition comprising cells and/or membrane portions of cells from an INB16 cell line (ATCC Deposit no. PTA-125809), wherein the cells and/or membrane portions of cells are rendered replication incompetent.

3. The biopharmaceutical composition of claim 2, comprising said cells from an INB16 cell line, wherein said cells are rendered replication incompetent by contact with mitomycin C.

4. The biopharmaceutical composition of claim 2, comprising said cells from an INB16 cell line, wherein said cells are rendered replication incompetent by first immobilizing said cells in a dry carbohydrate matrix and second dosing said cells while immobilized with a permeating ionizing radiation.

5. The biopharmaceutical composition of claim 4, wherein said dose comprises at least 2 Gy and up to 20 kGy.

6. A method for treating cancer, comprising: administering to a patient having said cancer a therapeutically effective amount of a priming tumor cell preparation for priming natural killer cells of the patient in vivo, the priming tumor cell preparation including: cells and/or membrane portions thereof derived from an INB16 cell line (ATCC Deposit no. PTA-125809), wherein the cells and/or membrane portions of the priming tumor cell preparation are rendered replication incompetent to prevent proliferation in the patient: whereby said patient is treated.

7. The method of claim 6, wherein the cancer comprises a solid tumor.

8. The method of claim 6, wherein the cancer comprises acute myeloid leukemia (AML).

9. The method of claim 6, wherein the cancer comprises myelodysplastic syndrome.

10. The method of claim 6, wherein the therapeutically effective amount comprises three weekly administrations of 1×10⁸ cells.
